(19) United States
(12) Patent Application Publication
(10) Pub. No.: US 2010/0063364 A1
(43) Pub. Date: Mar. 11, 2010

(54) INSTRUMENT INSERTION DEVICE

(76) Inventors: Frank Bonadio, County Wicklow (IE); John Butler, County Dublin (IE); Trevor Vaugh, County Offaly (IE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/525,256

(22) PCT Filed: Feb. 1, 2008
(86) PCT No.: PCT/IE08/00009

§ 371 Date: Nov. 20, 2009

Related U.S. Application Data
(60) Provisional application No. 60/898,716, filed on Feb. 1, 2007.

(51) Int. Cl. A61B 1/32

(52) U.S. Cl. 600/208

(57) ABSTRACT

An instrument insertion device (1) comprises a first seal (2), a second seal (3), a first proximal ring (4) for location externally of a wound opening, and a second proximal ring (5) for location externally of the wound opening. The first seal (2) may be a tricuspid valve defining a passageway (6) extending therethrough. An instrument (7) may be inserted through the passageway (6). The passageway (6) is movable from the closed configuration to the open configuration upon insertion of the instrument (7) through the passageway (6). The passageway (6) is biased towards the closed configuration, such that upon removal of the instrument (7) from the passageway (6), the passageway (6) moves automatically from the open configuration to the closed configuration. The second seal (3) may be a lipseal valve with a passageway (8) extending therethrough. An instrument (7) may be inserted through the passageway (8). The passageway (8) is biased towards the open configuration, such that upon removal of the instrument (7) from the passageway (8), the passageway (8) moves automatically from the sealed configuration to the open configuration.

### INTRODUCTION

This invention relates to an instrument insertion device.

### STATEMENTS OF INVENTION

According to the invention there is provided an instrument insertion device comprising:
a first seal member having a first passageway extending therethrough, through which an instrument is insertable; and
a second seal member having a second passageway extending therethrough, through which the instrument is insertable.

In one embodiment the first passageway is movable between a closed configuration to seal a wound opening, and an open configuration to facilitate insertion of an instrument through the first passageway.

The first passageway may be movable from the closed configuration to the open configuration upon insertion of an instrument through the first passageway.

In one case the first passageway is biased towards the closed configuration.

In one embodiment the first seal member has a protector to protect the seal member against damage by an instrument.

The first seal member may be divided into a number of sections and a protector is provided for at least some of the sections.

In one embodiment in the closed configuration, the first passageway is provided in the form of one or more slits through the first seal member.

The first passageway may be provided in the form of three slits through the first seal member.

In one embodiment the first seal member comprises a tricuspid valve.

In one embodiment the second passageway is movable between an open configuration, and a sealed configuration to seal around an instrument inserted through the second passageway.

The second passageway may be movable from the open configuration to the sealed configuration upon insertion of an instrument through the second passageway.

In one case the second passageway is biased towards the open configuration.

The second seal member may comprises a lipseal valve.

In one case the first seal member is located proximally of the second seal member.

In another case the first seal member is located distally of the second seal member.

The first seal member may be formed separately from the second seal member.

In one embodiment at least part of one of the seal members is longitudinally spaced apart from at least part of the other seal member.

One of the seal members may be movable relative to the other seal member.

The first seal member may be formed integrally with the second seal member.

In one case the device comprises a first proximal member for location externally of a wound opening. The seal member may be coupled to the first proximal member.

In one embodiment the seal member is fixed relative to the first proximal member.

In another embodiment the seal member is movable relative to the first proximal member. In this case the device may comprise a connecting member to connect the seal member to the first proximal member. The connecting member may be flexible.

The connecting member may comprise a sleeve.

In one embodiment the device comprises a retracting member for extending through a wound opening to retract laterally the sides of the wound opening.

The retracting member may be extendable through the wound opening in two layers.

In one case the retracting member is attached to the first proximal member.

The device may comprise a distal member for location internally of a wound opening. The retracting member may be coupled to the distal member. The retracting member may be looped around the distal member.

In one embodiment the device comprises a second proximal member for location externally of a wound opening. The second proximal member may be coupled to the first proximal member.

In one case the retracting member is extendable between the first proximal member and the second proximal member.

The two seal arrangement prevents loss of insufflation gases through the device, even as an instrument is being inserted or withdrawn.

### BRIEF DESCRIPTION OF THE DRAWINGS

The invention will be more clearly understood from the following description of some embodiments thereof, given by way of example only, with reference to the accompanying drawings, in which:

FIG. 1 is an exploded, isometric view of an instrument insertion device according to the invention;

FIG. 2 is a cut-away, isometric view of the device of FIG. 1;

FIGS. 3 to 5 are cross-sectional, side views of the device of FIG. 1, in use;

FIG. 6 is an isometric view of a part of the device of FIG. 1, in use;

FIG. 7 is a view similar to FIG. 2 of another instrument insertion device according to the invention;

FIG. 8 is a cut-away, isometric view of a part of the device of FIG. 7;

FIG. 9 is a view similar to FIG. 7 of a part of another instrument insertion device according to the invention;

FIGS. 10 to 12 are views similar to FIGS. 3 to 5 of another instrument insertion device according to the invention;

FIG. 13 is a cross-sectional, side view of a further instrument insertion device according to the invention;

FIG. 14 is a view similar to FIG. 13 of another instrument insertion device according to the invention;

FIG. 15 is a view similar to FIG. 14 of the device of FIG. 14, in use;

FIG. 16 is an isometric view of a part of another instrument insertion device according to the invention;

FIG. 17 is a side view of a further instrument insertion device according to the invention, in use;

FIG. 18 is an isometric view of the device of FIG. 17;

FIGS. 19 and 20 are views similar to FIGS. 17 and 18 of another instrument insertion device according to the invention;

FIG. 21 is an isometric view of another instrument insertion device according to the invention; and

FIGS. 22 to 24 are cross-sectional, side views of the device of FIG. 21, in use.

### DETAILED DESCRIPTION

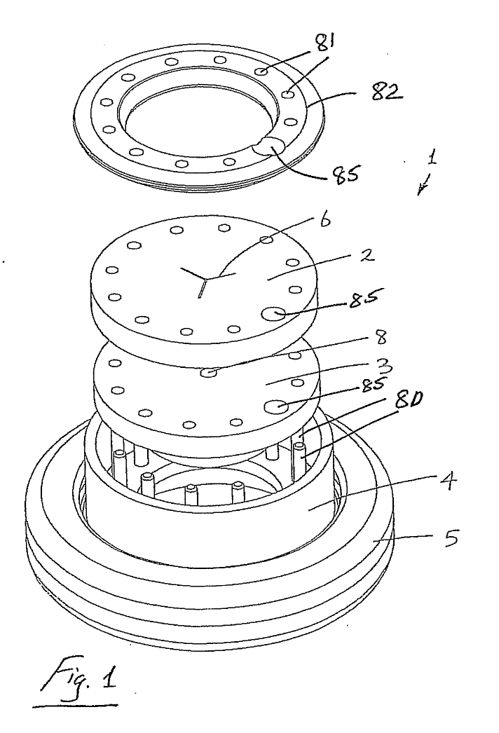

Referring to the drawings, and initially to FIGS. 1 to 6 thereof, there is illustrated an instrument insertion device 1 according to the invention. The device 1 comprises a first seal 2, a second seal 3, a first proximal ring 4 for location externally of a wound opening, and a second proximal ring 5 for location externally of the wound opening.

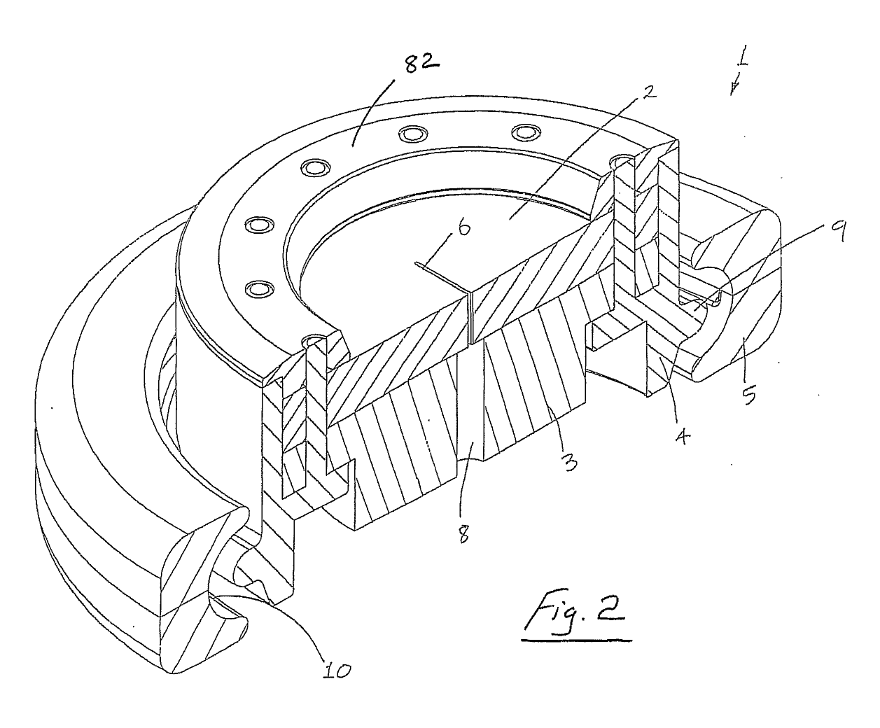

As illustrated in FIG. 2 the first seal 2 is located proximally of the second seal 3. Both of the seals 2, 3 are fixedly coupled to the first proximal ring 4. Spigots 80 extend upwardly from the ring 4 and holes 81 in an upper retaining plate 82 are aligned with the spigots to define a housing for the seals 2, 3. An insufflation port 86 extends through passageways 85 for entry of insufflation gas.

In this case the two seals 2, 3 are formed separately.

The second proximal ring 5 is located radially outwardly of the first proximal ring 4. The second proximal ring 5 is coupled to the first proximal ring 4 by means of a snap-fit engagement of an annular protrusion 9 on the first proximal ring 4 in an annular recess 10 of the seal proximal ring 5.

The first seal 2 has a passageway 6 extending therethrough. An instrument 7 may be inserted through the passageway 6. The passageway 6 is movable between a closed configuration (FIG. 3) to seal a wound opening, and an open configuration (FIG. 5) to facilitate insertion of the instrument 7 through the passageway 6. As illustrated in FIGS. 3 to 5, the passageway 6 is movable from the closed configuration to the open configuration upon insertion of the instrument 7 through the passageway 6. The passageway 6 is biased towards the closed configuration, such that upon removal of the instrument 7 from the passageway 6, the passageway 6 moves automatically from the open configuration to the closed configuration.

In this case the first seal 2 comprises a tricuspid valve. As illustrated in FIG. 1, when the passageway 6 is in the closed configuration, the passageway 6 is in the form of three slits through the first seal 2.

The second seal 3 has a passageway 8 extending therethrough. An instrument 7 may be inserted through the passageway 8. The passageway 8 is movable between an open configuration (FIG. 3), and a sealed configuration (FIG. 5) to seal around the instrument 7 inserted through the passageway 8. As illustrated in FIGS. 3 to 5, the passageway 8 is movable from the open configuration to the sealed configuration upon insertion of the instrument 7 through the passageway 8. The passageway 8 is biased towards the open configuration, such that upon removal of the instrument 7 from the passageway 8, the passageway 8 moves automatically from the sealed configuration to the open configuration.

In this case the second seal 3 comprises a lipseal valve.

As illustrated in FIG. 6, when the instrument 7 is inserted through the first seal 2, there may be one or more spaces between the instrument 7 and the sides of the passageway 6. By providing the second seal 3 in addition to the first seal 2, this arrangement prevents any gas leakage through these spaces because the second seal 3 seals tightly around the instrument 7 inserted through the passageway 8.

FIG. 1 illustrates the trislit (zero) valve 2 and the lipseal 3 which are formed as two separate components.

FIG. 2 illustrates the trislit (zero) valve 2 and the gel lipseal 3, which are not fixed together.

FIG. 3 illustrates the clip applier 7, the tricusp valve 2, and the lipseal 3.

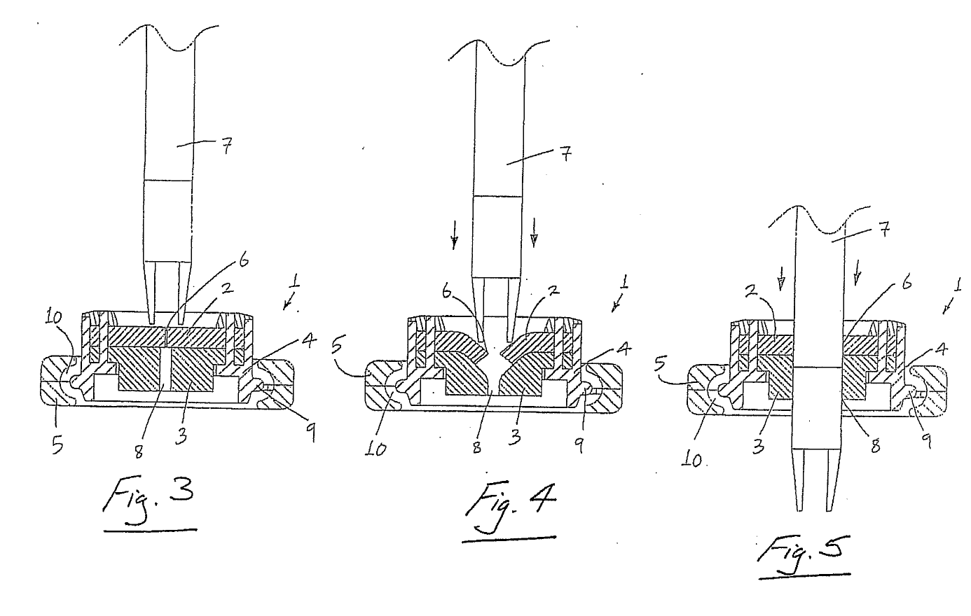

FIG. 4 illustrates the three flaps pushed down and outwardly by the instrument tip. This causes the lipseal 3 to open up easier passage through the instrument 7.

FIG. 5 illustrates the leak paths at the three corners of the tricusp valve 2. The seal is achieved at the lipseal 3.

FIG. 6 illustrates the laparoscopic instrument shaft 7, leak paths with the instrument 7 in place, and the trislit valve 2.

In use a wound opening is created in a tissue, and the device 1 is positioned externally of the wound opening. To access the wound opening and/or the interior of the wound opening with the instrument 7, the instrument 7 is advanced distally to engage against the first seal 2. As the instrument 7 is inserted through the passageway 6, this causes the passageway 6 to move from the closed configuration to the open configuration. Similarly as the instrument 7 is inserted through the passageway 8, this causes the passageway 8 to move from the open configuration to the sealed configuration (FIG. 5). The instrument 7 may then be advanced further to access the wound opening and/or the interior of the wound opening.

To remove the instrument 7 from the wound opening and/or the interior of the wound opening, the instrument 7 is retraced proximally. The biassing nature of the second seal 3 causes the passageway 8 to automatically move from the sealed configuration to the open configuration. Similarly the biassing nature of the first seal 2 causes the passageway 6 to automatically move from the open configuration to the closed configuration.

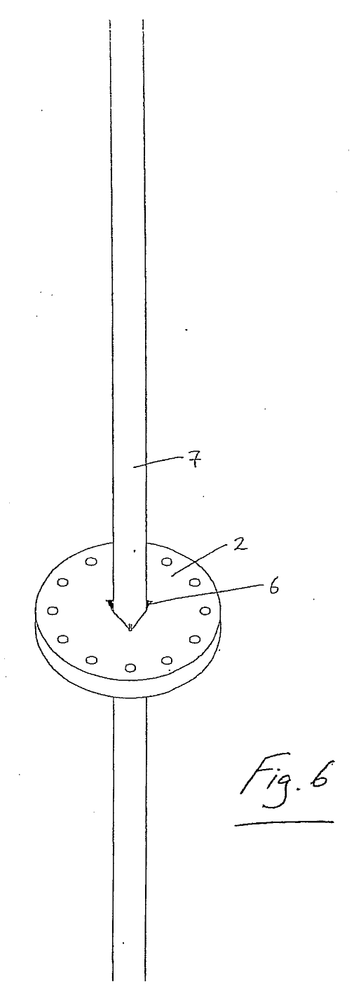

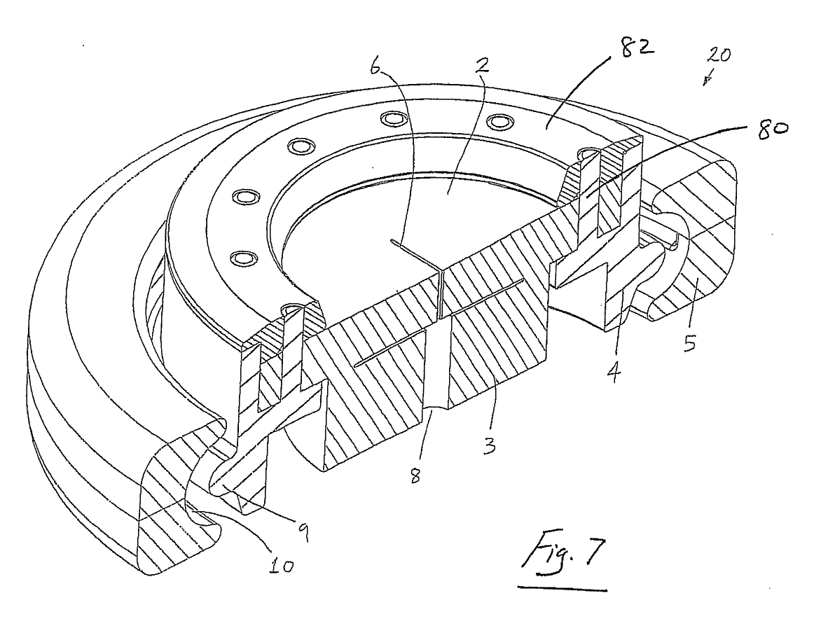

In the instrument access device 20 of FIGS. 7 and 8, the first seal 2 is formed integrally with the second seal 3.

FIG. 7 is a section view of the trislit valve 2, the lipseal 3, and the annular connection between the trislit valve 2 and the lipseal 3.

FIG. 8 illustrates the two valves 2, 3 moulded from one material. FIG. 8 illustrates the trislit (zero) valve 2, the lipseal 3, and the annular connection between the two valves 2, 3.

In the embodiment of FIG. 9, part of the first seal 2 is longitudinally spaced apart from part of the second seal 3.

The first and second seals may be both formed of a gel material. The gel material may include an elastomer, such as silicone or latex. The gel material may also include an oil, and/or a foam.

In one case the seal is of a gelatinous elastomeric material. An extensive review of gelatinous elastomeric materials is included in U.S. Pat. No. 5,994,450 (Pierce), the entire contents of which are incorporated herein by reference. One such group of gelatinous elastomers may comprise a triblock copolymer A-B-A wherein A is selected from the group consisting of monoalkenylarene polymers and B is a hydrogenated polymer including a plurality of isoprene monomers and a plurality of butadiene monomers. The material includes a plasticiser which may be selected from the group consisting of naturally derived oils, synthetic oils and liquid oligomers. For the device of this embodiment of the invention the gelatinous elastomeric material is formulated to have high tear strength and high flexibility.

The materials required to form a suitable gel material are available, for example, from the company Edizone L.C. of Utah, USA. The gel material preferably has the properties of high tear strength to resist tearing and high flexibility to enable the seal to be retracted for passage of an instrument through the passageway into the abdomen 4.

The first seal 2 may be of a gel with stiffness “X”, and the second seal 3 may be of a gel with stiffness “Y”. Gel X may be stiffer than gel Y. FIG. 9 illustrates the trislit valve 2, the lipseal 3, and the annular connection between gel X and gel Y. The connection may be glue or heat welded.

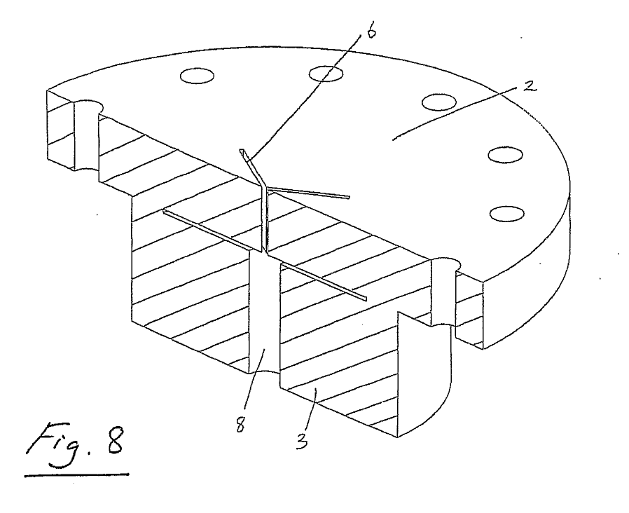

In the instrument access device 30 of FIGS. 10 to 12, the entire first seal 2 is longitudinally spaced apart from the entire second seal 3.

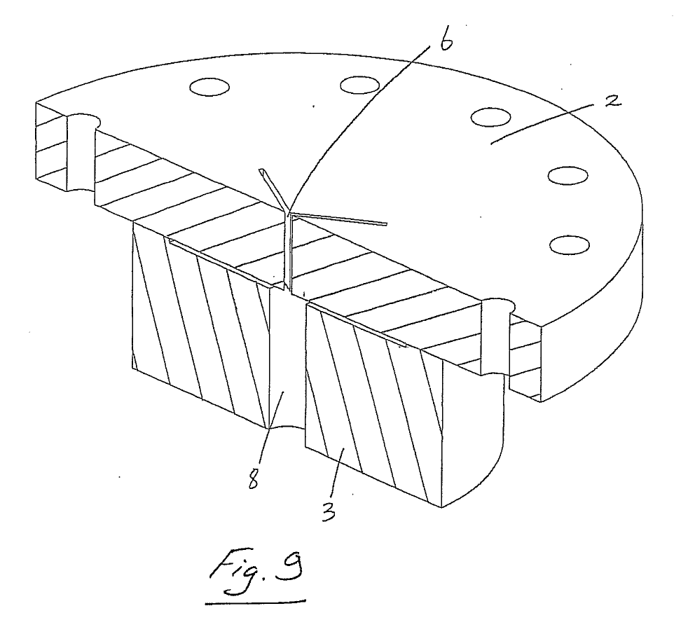

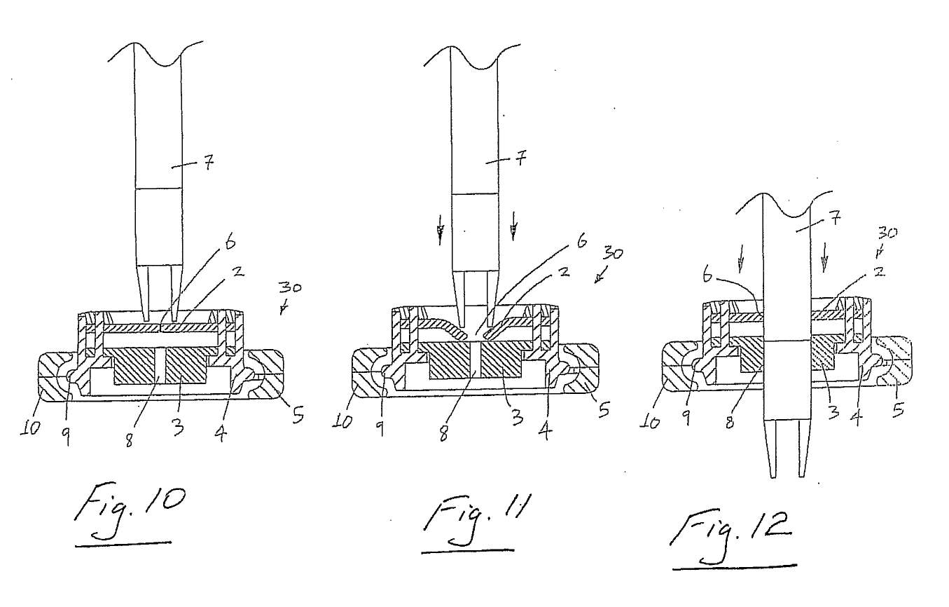

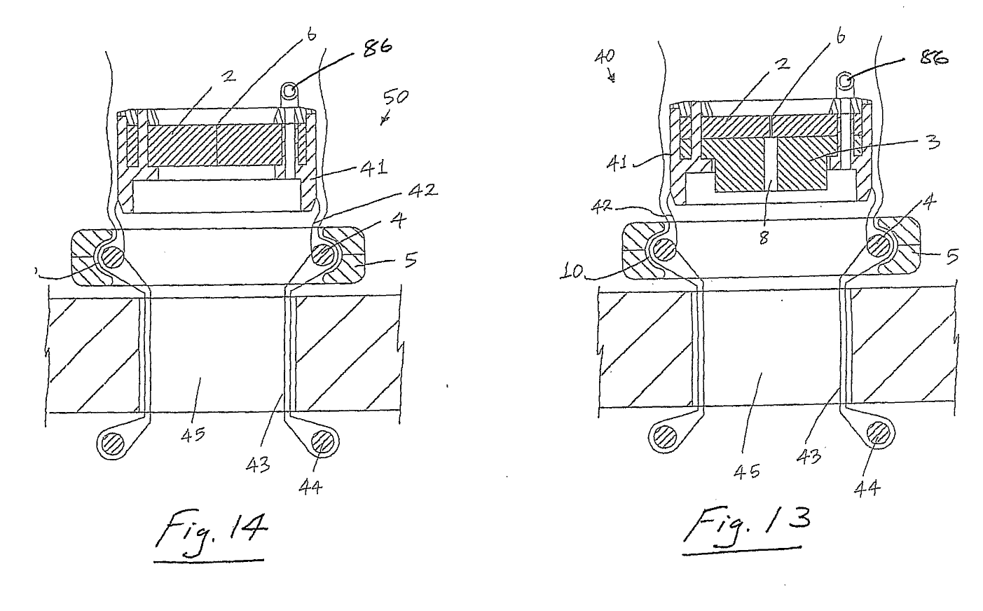

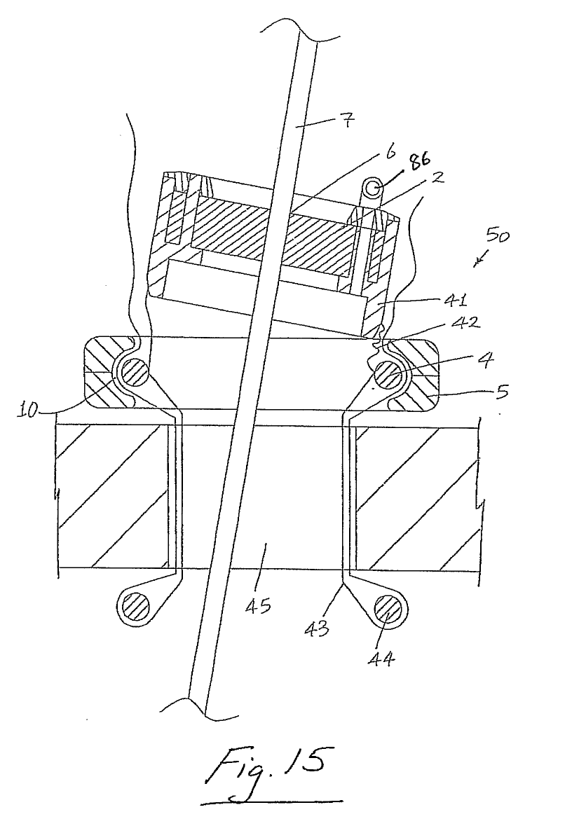

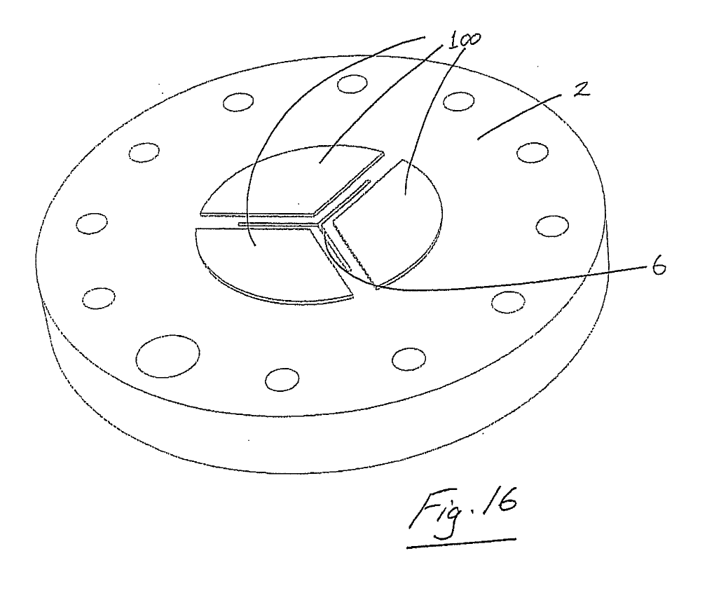

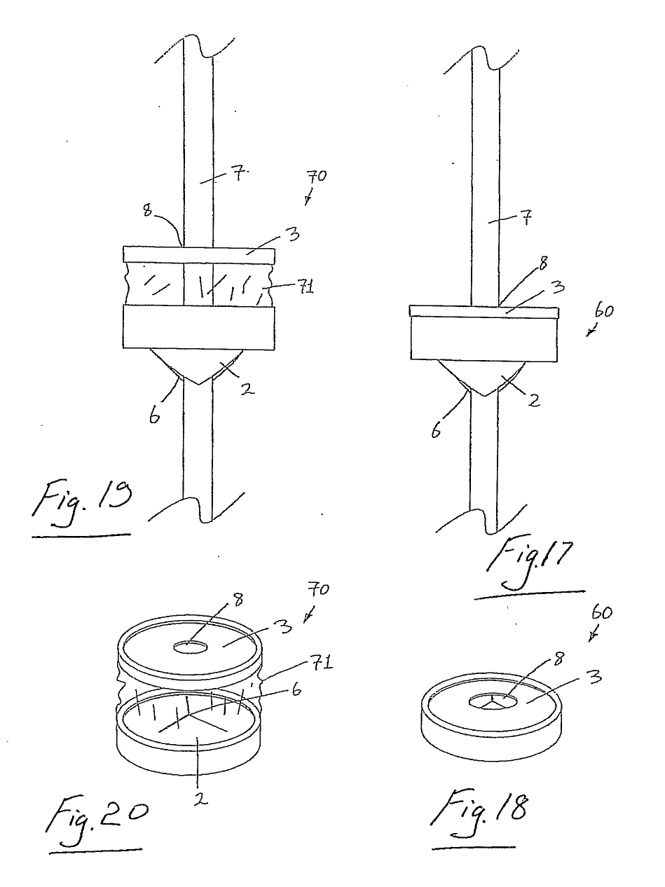

FIG. 10 illustrates the instrument 7, for example a clip applier, the tricusp valve 2, a gap, and then the lipseal 3. FIG. 11 illustrates the clip applier parting the tricusp valve 2. FIG. 12 illustrates the seal now achieved by the lipseal 3.

The retractor base used in association with the valve system can be of any suitable construction such as the retractors described in our US patent application published under No. 2001/0037053A, and/or U.S. Pat. No. 6,582,364, and/or US patent application published under No. 2005/0090717A the whole contents of all of which are incorporated herein by reference.

In the instrument access device 40 of FIG. 13, the device 40 comprises a housing 41 and a flexible sleeve 42 to connect the housing 41 to the first proximal ring 4. Both of the seals 2, 3 are fixedly coupled to the housing 41. The housing 41 is movable relative to the first proximal ring 4.

In addition the device 40 comprises a retracting sleeve 43 and a distal ring 44 for location internally of the wound opening 45.

The retracting sleeve 43 extends through the wound opening 45 in two layers to retract laterally the sides of the wound opening 45. One end of the retracting sleeve 43 is attached to the first proximal ring 4. The retracting sleeve 43 is coupled to the distal ring 44 by being looped around the distal ring 44. The other end of the retracting sleeve 43 extends proximally between the first proximal ring 4 and the second proximal ring 5.

FIG. 13 illustrates the lipseal valve 3, the tricusp valve 2, and an insufflation port, and the floating sleeve 42.

FIGS. 14 and 15 illustrate another instrument insertion device 50 which is similar to the device 40 of FIG. 13.

FIG. 14 illustrates the gel valve, the insufflation port, and the floating sleeve 42.

FIG. 15 illustrates the floating sleeve 42. The floating sleeve 42 reduces the friction on the instrument shaft when it is tilted off-axis.

FIG. 16 illustrates the tricusp valve 2, with a protector provided by pads 100 to deflect the instrument tips, for example a clip applier, and to protect the gel to prevent the instrument tips damaging or embedding in the gel.

In the instrument access device 60 of FIGS. 17 and 18, the first seal 2 is located distally of the second seal 3.

In the instrument access device 70 of FIGS. 19 and 20, the first seal 2 is connected to the second seal 3 by means of a flexible sleeve 71. The first seal 2 is movable relative to the second seal 3.

FIGS. 17 and 18 illustrate the lipseal valve 3 and the elastic/rubber/gel valve 3 with three slits. The tricuspid valve 2 forms an airtight seal before the instrument 7 is inserted, and the lipseal 3 after.

FIG. 19 illustrates the floating lipseal 3 of gel/rubber, the film/gel 71, and the lower valve 2 which opens when the instrument 7 is inserted.

FIG. 20 illustrates the lipseal 3 floating, and the simple slits forming the valve 2.

In FIG. 18 the lipseal 3 is fixed.

In the instrument access device 80 of FIGS. 21 to 24 the first seal 2 is connected to the second seal 3 by means of movable coupling 81. The first seal 2 is movable relative to the second seal 3.

In FIGS. 21 to 24 this valve consists of the floating/moving lipseal 3 and the simple elastic/gel/rubber valve 2 with three slits to allow it to open. The lipseal 3 could also be made from only elastic material.

FIG. 21 illustrates the over hang which stops the floating lipseal 3 from inverting, and space for an insufflation cup/system.

FIG. 22 illustrates the floating lipseal 3 with an opening, and the three slit valve 2.

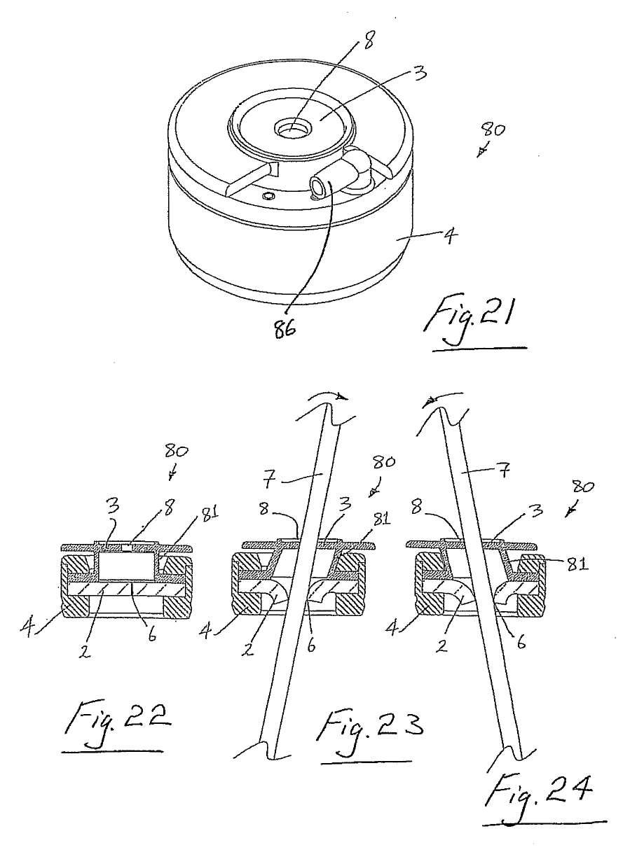

FIG. 23 illustrates the three slit valve 2 which opens when the instrument 7 is inserted.

FIG. 24 illustrates the floating lipseal 3 which allows the instrument 7 to move without air loss.

The invention is not limited to the embodiments hereinbefore described, with reference to the accompanying drawings, which may be varied in construction and detail.

1. An instrument insertion device comprising:
a first seal member having a first passageway extending therethrough, through which an instrument is insertable; and
a second seal member having a second passageway extending therethrough, through which the instrument is insertable.

2. A device as claimed in claim 1 wherein the first passageway is movable between a closed configuration to seal a wound opening, and an open configuration to facilitate insertion of an instrument through the first passageway.

3. A device as claimed in claim 2 wherein the first passageway is movable from the closed configuration to the open configuration upon insertion of an instrument through the first passageway.

4. A device as claimed in claim 2 wherein the first passageway is biased towards the closed configuration.

5. A device as claimed in claim 1 wherein the first seal member has a protector to protect the seal member against damage by an instrument.

6. A device as claimed in claim 4 wherein the first seal member is divided into a number of sections and a protector is provided for at least some of the sections.

7. A device as claimed in claim 2 wherein in the closed configuration, the first passageway is provided in the form of one or more slits through the first seal member.

8. A device as claimed in claim 7 wherein the first passageway is provided in the form of three slits through the first seal member.

9. A device as claimed in claim 8 wherein the first seal member comprises a tricuspid valve.

10. A device as claimed in claim 1 wherein the second passageway is movable between an open configuration, and a sealed configuration to seal around an instrument inserted through the second passageway.

11. A device as claimed in claim 10 wherein the second passageway is movable from the open configuration to the sealed configuration upon insertion of an instrument through the second passageway.

12. A device as claimed in claim 10 wherein the second passageway is biased towards the open configuration.

13. A device as claimed in claim 10 wherein the second seal member comprises a lipseal valve.

14. A device as claimed in claim 1 wherein the first seal member is located proximally of the second seal member.

15. A device as claimed in claim 1 wherein the first seal member is located distally of the second seal member.

16. A device as claimed in claim 1 wherein the first seal member is formed separately from the second seal member.

17. A device as claimed in claim 1 wherein at least part of one of the seal members is longitudinally spaced apart from at least part of the other seal member.

18. A device as claimed in claim 1 wherein one of the seal members is movable relative to the other seal member.

19. A device as claimed in claim 1 wherein the first seal member is formed integrally with the second seal member.

20. A device as claimed in claim 1 wherein the device comprises a first proximal member for location externally of a wound opening.

21. A device as claimed in claim 20 wherein the seal member is coupled to the first proximal member.

22. A device as claimed in claim 21 wherein the seal member is fixed relative to the first proximal member.

23. A device as claimed in claim 21 wherein the seal member is movable relative to the first proximal member.

24. A device as claimed in claim 23 wherein the device comprises a connecting member to connect the seal member to the first proximal member.

25. A device as claimed in claim 24 wherein the connecting member is flexible.

26. A device as claimed in claim 25 wherein the connecting member comprises a sleeve.

27. A device as claimed in claim 20 wherein the device comprises a retracting member for extending through a wound opening to retract laterally the sides of the wound opening.

28. A device as claimed in claim 27 wherein the retracting member is extendable through the wound opening in two layers.

29. A device as claimed in claim 27 wherein the retracting member is attached to the first proximal member.

30. A device as claimed in claim 27 wherein the device comprises a distal member for location internally of a wound opening.

31. A device as claimed in claim 30 wherein the retracting member is coupled to the distal member.

32. A device as claimed in claim 31 wherein the retracting member is looped around the distal member.

33. A device as claimed in claim 27 wherein the device comprises a second proximal member for location externally of a wound opening.

34. A device as claimed in claim 33 wherein the second proximal member is coupled to the first proximal member.

35. A device as claimed in claim 33 wherein the retracting member is extendable between the first proximal member and the second proximal member.

36. (canceled)
